SEQUENCE LISTING (2 sequences)

SEQ ID NO: 1 - DNA, 20 bp, Artificial sequence
  Sense primer
ccccaccccattactgtacc

SEQ ID NO: 2 - DNA, 20 bp, Artificial sequence
  Antisense primer
cccagcacacccttcttcct

(19) United States
(12) Patent Application Publication
(10) Pub. No.: US 2023/0167500 A1
(43) Pub. Date: Jun. 1, 2023

(54) SUDDEN CARDIAC DEATH SUSCEPTIBILITY DETECTION KIT BASED ON INSERTION/DELETION POLYMORPHIC SITE OF COX10 GENE

(71) Applicant: SOOCHOW UNIVERSITY, Suzhou (CN)

(72) Inventors: Yan HE, Suzhou (CN); Zhenzhen YANG, Suzhou (CN); Huan YU, Suzhou (CN); Yuzhen GAO, Suzhou (CN)

(21) Appl. No.: 17/762,065

(22) PCT Filed: Oct. 21, 2020
(86) PCT No.: PCT/CN2020/122331

§ 371 Date: Mar. 20, 2022

(30) Foreign Application Priority Data
Oct. 10, 2020 (CN) 202011079845.6

(51) Int. Cl. C12Q 1/6883

(52) U.S. Cl. CPC C12Q 1/6883; C12Q 2600/156

(57) ABSTRACT

The invention provides a sudden cardiac death susceptibility detection kit based on an insertion/deletion polymorphic site of COX10 gene. A specific primer pair contained in the kit is designed for the insertion/deletion site rs397763766 on the COX10 gene. DNA fragments containing the site are specifically amplified. Different genotypes are recognized by detecting the mobility of fragments different in length in capillary electrophoresis. In combination with findings in a case-control study, it is believed that an individual carrying a deletion-type allele at the insertion/deletion site rs397763766 of COX10 gene in the detected DNA is SCD susceptible. Therefore, the SCD susceptibility of an individual is predicted by detecting the genotype of the insertion/deletion site rs397763766 on the COX10 gene in the individual. It is confirmed that a two-base (CT) insertion/deletion polymorphism (rs397763766) located in the 3′UTR of the COX10 gene is significantly associated with the risk of suffering from SCD.

### FIELD OF THE INVENTION

The present invention relates to the field of detection technologies, and more particularly to a sudden cardiac death susceptibility detection kit based on an insertion/deletion polymorphic site of COX10 gene.

### DESCRIPTION OF THE RELATED ART

Sudden cardiac death (SCD) refers to sudden unexpected death caused by heart disorder, which is manifested as sudden consciousness loss, and circulatory and respiratory arrests that occur in a short period of time. “A short period of time” is defined as less than 1 hour when there is a witness, and 24 hours when there is no witness. Epidemiological research shows that 40.7 out of 100,000 people died of SCD in China each year. Considering the huge population in China, there is a huge amount of SCD patients. Previous studies have shown that SCD patients mostly died of coronary atherosclerotic heart disease or fatal arrhythmia. The former is common in elderly population, while the latter is common in young population. In view of the difficulty to find exact cause of death by postmortem autopsy and histological examination, it is urgent to discover the molecular mechanism of SCD and find a genetic marker of SCD so as to accurately diagnose SCD.

In the field of SCD diagnosis, studies mainly focused on analysis of the correlation between mutations in the coding region of important protein-coding genes and sudden death. However, the number of related mutations is limited, and the extremely low gene frequency in the general population cannot fully explain the relatively high incidence of sudden death. With the development of genome-wide association study and next-generation sequencing technology, more and more genetic markers have been found to be involved in the regulation of SCD. The identification of genetic markers for SCD is helpful to further understand the underlying mechanism of SCD, optimize the risk stratification of SCD, and provide a powerful theoretical basis for molecular diagnosis and prevention. Considering the significant role of 3′UTR in the regulation of gene expression, the attention is focused on the genetic polymorphisms of 3′UTR, and the correlation between genetic polymorphisms of 3′UTR in related genes and SCD susceptibility is analyzed by control—case study, which allows for the establishment of a genetic polymorphism detection system that can be used to assess the risk of suffering from SCD.

Cytochrome c oxidase assembly factor heme A: farnesyltransferase COX10 is a functional subunit on the mitochondrial oxidative respiratory chain transcribed by nuclear genes. It is a key enzyme for the production of heme A prosthetic group. Previous studies have shown that abnormal expression of COX10 protein can cause energy production disorder in the body, affect the contractile function of cardiomyocytes, and lead to a series of changes in cardiac function.

In the prior art, there is no report on the research of the correlation between the insertion/deletion polymorphism at the site rs397763766 and SCD, and no report on the prediction of SCD susceptibility by detecting the insertion/deletion polymorphic site in the 3′UTR of the COX10 gene.

### SUMMARY OF THE INVENTION

In order to solve the above problem, the present invention provides a sudden cardiac death susceptibility detection kit based on the insertion/deletion polymorphic site rs397763766 of COX10 gene, which can be used to assess the SCD susceptibility of an individual.

A first object of the present invention is to provide a sudden cardiac death susceptibility detection kit based on an insertion/deletion polymorphic site of COX10 gene, wherein the kit is used for detecting the genotype of the site rs397763766 on the COX10 gene.

Preferably, the sudden cardiac death susceptibility detection kit comprises a specific primer pair for detecting the site rs397763766 on the COX10 gene, and a component for PCR amplification and capillary electrophoresis.

Preferably, the specific primer pair comprises a sense primer and an antisense primer; and the nucleotide sequence of the sense primer is shown in SEQ ID NO:1, and the nucleotide sequence of the antisense primer is shown in SEQ ID NO:2:

SEQ ID NO: 1: 5′-CCCCACCCCATTACTGTACC-3′
SEQ ID NO: 2: 5′-CCCAGCACACCCTTCTTCCT-3′

Preferably, the Tm value of the sense primer is 62° C., and the Tm value of the antisense primer is 62° C.

Preferably, a fluorescent marker is provided at the 5′-terminus of the specific primer pair.

In the present invention, the fluorescently-labeled specific primers are designed for the insertion/deletion site rs397763766 on the COX10 gene, with which fragments containing the insertion/deletion site can be specifically amplified. A fluorescent dye is labeled on the 5′-terminus of the oligonucleotide primers by fluorescence labeling technology. The strand of the product from PCR amplification carries the fluorescent dye labeled on the primer. Those skilled in the art can understand the experimental principles. Conventional synthesis techniques can be used to synthesize the specific primer pair. In a preferred embodiment, the SCD susceptibility detection kit comprises the primer pair comprising sequences shown in SEQ ID NO: 1 and SEQ ID NO: 2. It should be noted that the primers of the present invention are not limited to such pair of primers.

Preferably, the component for PCR amplification and capillary electrophoresis comprise: Taq DNA polymerase, dNTP mixture, MgCl₂ solution, PCR reaction buffer and deionized water.

Preferably, the sudden cardiac death susceptibility detection kit comprises 50 μM specific primer pair, 2.5 U/μl Taq DNA polymerase, 2.5 mM dNTP mixture, 25 mM MgCl₂ solution, 10× PCR reaction buffer and deionized water.

Preferably, a product from the PCR amplification is genotyped by capillary electrophoresis.

A second object of the present invention is to provide use of the sudden cardiac death susceptibility detection kit in detecting the genotype of the site rs397763766 on the COX10 gene.

Preferably, the use comprises obtaining a PCR amplified product by fluorescence-labeled PCR amplification; and genotyping the amplified product by capillary electrophoresis.

Molecular biology research shows that different allele genotypes of the insertion/deletion polymorphic site rs397763766 in the 3′UTR of COX10 gene can affect the transcription activity of the COX10 gene. The transcription activity of COX10 is relatively high in an individual carrying the deletion-type allele. In addition, case-control study on SCD in a certain scale of population demonstrates that the deletion-type allele of the insertion/deletion polymorphism was positively correlated with the occurrence of SCD, and even factors such as age and gender are adjusted, such correlation still exists. Therefore, it is believed that the insertion/deletion polymorphism can be used to assess the SCD susceptibility in an individual.

The beneficial effects of the present invention are as follows.

The specific primer pair contained in the kit of the present invention is designed for the insertion/deletion site rs397763766 of COX10 gene. DNA fragments containing the site can be specifically amplified. Different genotypes are recognized by detecting the mobility of fragments different in length in capillary electrophoresis. In combination with findings in a case-control study, it is believed that an individual carrying a deletion-type allele at the insertion/deletion site rs397763766 of COX10 gene in the detected DNA is SCD susceptible. Therefore, in the present invention, the SCD susceptibility of an individual can be predicted by detecting the genotype of the insertion/deletion site rs397763766 on the COX10 gene in the individual. The inventors confirm, for the first time, that a two-base (CT) insertion/deletion polymorphism (rs397763766) located in the 3′UTR of the COX10 gene is significantly associated with the risk of suffering from SCD. The frequency distribution of this polymorphism in Asian population is 0.62 for the insertion type and 0.38 for the deletion type.

### BRIEF DESCRIPTION OF THE DRAWINGS

FIG. 1 is a schematic diagram of gene sequencing and genotyping by SDS-PAGE gel electrophoresis.

### DETAILED DESCRIPTION OF THE PREFERRED EMBODIMENTS

The present invention will be further described below in conjunction with specific examples, so that those skilled in the art can better understand and implement the present invention. It should be noted that examples provided herein are not intended to limit the present invention.

### Example 1: Use of a Detection Kit

Step 1: Extraction of DNA Template

A blood genomic DNA extraction system (non-spin column type) was used to extract genomic DNA from peripheral blood.

Step 2: PCR Reaction-Replication of the Target Fragment

A PCR detection kit that can detect SCD susceptibility was used, where the kit comprises the following primers:

SEQ ID NO: 1: 5′-CCCCACCCCATTACTGTACC -3′,
with a Tm value of 62° C.;
SEQ ID NO: 2: 5′-CCCAGCACACCCTTCTTCCT -3′,
with a Tm value of 62° C.;

With the primer pair, a fragment of the COX10 gene containing the insertion/deletion polymorphic site rs397763766 could be specifically amplified.

The total volume of the PCR reaction system was 10 μl, including: 1 μl DNA template, 50 μM specific primer pair for 0.04 μl each, 0.08 μl of 2.5 U/μl Taq DNA polymerase; 0.2 μl of 2.5 mM dNTP mixture; 0.6 μl of 25 mM MgCl₂ solution; 1 μl of 10× PCR reaction buffer; and supplementary amount of deionized water. The reaction was carried out on Eppendorf Mastercycler nexus PCR machine, under the reaction conditions of: 94° C. for 3 min; then 30 PCR cycles of 94° C. for 30 s, 62° C. for 30 s, and 72° C. for 1 min; and final 72° C. for 5 min.

Step 3: Insertion/Deletion Genotype Analysis

After amplification, the product was isolated by capillary electrophoresis using ABI 3500 gene sequencer to obtain the genotype of the tested individual, and an explanation was given by a skilled person.

The difference in gene frequency at the site between the control group and the SCD group and the OR value for assessing the risk of developing disease are shown in Table 1.

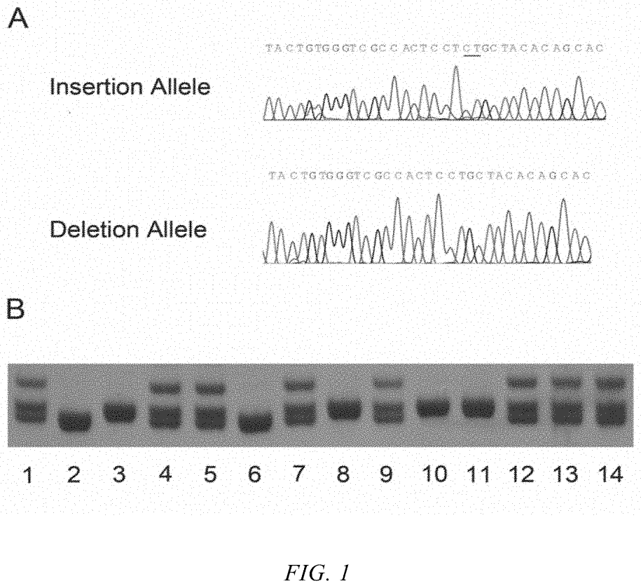

The gene sequencing and genotyping by SDS-PAGE gel electrophoresis are shown in FIG. 1. FIG. 1A is a schematic diagram of the sequencing result for the template strand, and the underlined content corresponds to the two-base insertion and deletion of the coding strand at rs397763766; FIG. 1B is an electrophoregram for the product obtained by the PCR amplification system of the present invention against 14 DNA samples from different individuals. 3, 8, 11 and 12 are insertion-type homozygotes, 2 and 6 are deletion-type homozygotes, and the rest are heterozygotes.

TABLE 1
The correlation between the polymorphic site rs397763766 and the risk of SCD

| Genetic | | Case | Control | | | |
|---|---|---|---|---|---|---|
| Model | Genotype | Group (%) | Group (%) | OR(95% CI)^{a} | | P value |
| Co- | Insertion/insertion | 38(24.05) | 206(37.87) | 1.00 | (reference) | |
| dominant | Insertion/deletion | 83(52.53) | 263(48.35) | 1.71 | (1.12-2.62) | 1.27 × 10⁻³ |
| Model | Deletion/deletion | 37(23.42) | 75(13.78) | 2.67 | (1.58-4.52) | 1.76 × 10⁻⁴ |
| | P_{trend} | | | | | 7.93 × 10⁻⁴ |
| Dominant | Insertion/insertion | 64(24.05) | 344(37.87) | 1.00 | (reference) | |
| Model | Insertion/deletion + | 95(75.95) | 324(62.13) | 1.93 | (1.29-2.88) | 1.33 × 10⁻³ |
| | deletion/deletion | | | | | |
| Additive | Insertion-type | 197(50.32) | 958(62.04) | 1.00 | (reference) | |
| Model | allele | | | | | |
| | Deletion-type | 121(49.68) | 378(37.96) | 1.61 | (1.25-2.07) | 1.87 × 10⁻⁴ |

allele

Note:
^{a}calibrated according to the age and gender.
OR: odds ratio (risk rate);
CI: confidence interval

As shown in Table 1, in a co-dominant model, the risk of suffering from SCD in an individual with a deletion/deletion genotype in a confidence interval of 95% is 2.67 times that in an individual with an insertion/insertion genotype, and the risk of suffering from SCD in an individual with an insertion/deletion genotype is 1.71 times that in an individual with an insertion/insertion genotype. In a dominant model, the risk of suffering from SCD in an individual with an insertion/deletion or deletion/deletion genotype is 1.93 times that in an individual with insertion/insertion genotype. Finally, the additive model analysis shows that the risk of SCD in an individual with deletion-type allele is 1.61 times that in an individual with insertion-type allele.

The above-mentioned embodiments are merely preferred embodiments provided for fully illustrating the present invention. The protection scope of the present invention is not limited thereto. Equivalent substitutions or alterations made by those skilled in the art on the basis of the present invention are all contemplated in the protection scope of the present invention as defined by the claims.

1. A sudden cardiac death susceptibility detection kit based on an insertion/deletion polymorphic site of COX10 gene, wherein the kit is used for detecting the genotype of the site rs397763766 on the COX10 gene.

2. The sudden cardiac death susceptibility detection kit according to claim 1, wherein the sudden cardiac death susceptibility detection kit comprises a specific primer pair for detecting the site rs397763766 on the COX10 gene, and a component for PCR amplification and capillary electrophoresis.

3. The sudden cardiac death susceptibility detection kit according to claim 2, wherein the specific primer pair comprises a sense primer and an antisense primer; and the nucleotide sequence of the sense primer is shown in SEQ ID NO:1, and the nucleotide sequence of the antisense primer is shown in SEQ ID NO:2:
SEQ ID NO: 1: 5′-CCCCACCCCATTACTGTACC-3′
SEQ ID NO: 2: 5′-CCCAGCACACCCTTCTTCCT-3′

4. The sudden cardiac death susceptibility detection kit according to claim 3, wherein the Tm value of the sense primer is 62° C., and the Tm value of the antisense primer is 62° C.

5. The sudden cardiac death susceptibility detection kit according to claim 2, wherein a fluorescent marker is provided at the 5′-terminus of the specific primer pair.

6. The sudden cardiac death susceptibility detection kit according to claim 2, wherein the component for PCR amplification and capillary electrophoresis comprise: Taq DNA polymerase, dNTP mixture, MgCl₂ solution, PCR reaction buffer and deionized water.

7. The sudden cardiac death susceptibility detection kit according to claim 2, wherein the sudden cardiac death susceptibility detection kit comprises 50 μM specific primer pair, 2.5 U/μl Taq DNA polymerase, 2.5 mM dNTP mixture; 25 mM MgCl₂ solution, 10×PCR reaction buffer; and deionized water.

8. The sudden cardiac death susceptibility detection kit according to claim 2, wherein a product from the PCR amplification is genotyped by capillary electrophoresis.

9. Use of the sudden cardiac death susceptibility detection kit according to claim 1 in detecting the genotype of the site rs397763766 on the COX10 gene.

10. The use according to claim 9, wherein the use comprises obtaining a PCR amplified product by fluorescence-labeled PCR amplification; and genotyping the amplified product by capillary electrophoresis.
